(19) United States
(12) Patent Application Publication
(10) Pub. No.: US 2011/0183385 A1
(43) Pub. Date: Jul. 28, 2011

(54) METHOD OF EXTRACTION AND YIELD-UP OF TRICYCLO COMPOUNDS BY ADDING A SOLID ADSORBENT RESIN AS THEIR CARRIER IN FERMENTATION MEDIUM

(76) Inventors: Jae Jong Kim, Daejeon (KR); Si Kyu Lim, Daegu (KR); Mi Ok Lee, Deajeon (KR); Sang Myoun Lim, Daejeon (KR); Bo-mi Lee, Nonsan-si (KR); Dong Hwan Kim, Daejeon (KR); Jeoung Hyun, Cheongju-si (KR); Keum Soon Lee, Daejeon (KR)

(21) Appl. No.: 12/672,479

(22) PCT Filed: May 6, 2008
(86) PCT No.: PCT/KR08/02520

§ 371 Date: Feb. 5, 2010

(30) Foreign Application Priority Data
Aug. 17, 2007 (KR) 10-2007-0082642

(51) Int. Cl. C12P 17/18

(52) U.S. Cl. 435/119

(57) ABSTRACT

The present invention relates to a method of fermenting and purifying tricyclo compounds, specifically FK506 and/or FK520, and more particularly, to a method of purifying tricyclo compounds by adding a hydrophobic absorbent resin as a carrier in culturing FK506 and/or FK520 producing bacteria.

### TECHNICAL FIELD

The present invention relates to tricyclo compounds, and more particularly to a method of fermenting and purifying FK506 and/or FK520.

### BACKGROUND ART

The present invention relates to tricyclo compounds, as disclosed in U.S. Pat. No. 4,894,366, and more particularly to a method for improving production of tacrolimus (FK506) and ascomycin (FK520) and purifying the same.

In general, tricyclo compounds are biologically active materials that are secondary metabolites produced by microorganisms, particularly actinomyces sp. and display antifungal activity and inimunosupressive activity. The general structure of the tricyclo compounds is shown in Chemical Formula 1 below.

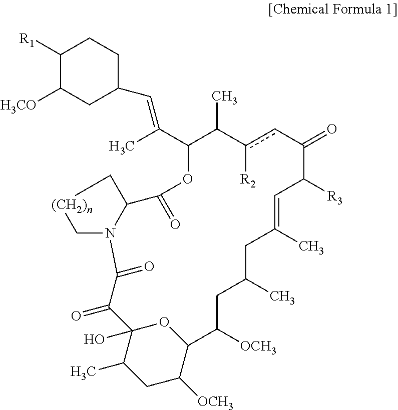

(In the above Chemical Formula, R1 is a hydroxyl group or a protected hydroxyl group. R2 is hydrogen, a hydroxyl group or a protected hydroxyl group. R3 is a methyl, ethyl, propyl or allyl group. In addition, “n” is 1 or 2 and the double solid and dotted line represent a single or a double bond.)

Important examples of tricyclo compounds include FK506, FK520 and rapamycin and the likes are known to have a similar structure. Particularly, FK506 has superior immunosuppressive activity to cyclosporin A, as reported by Kino et al. in 1987 (Hantanaka, H., M. Iwai, T. Kino. T. Goto, and M. Okuhara. 1988. J. Antibiot. 41: 1586-1591; Kino, T., H. Hantanaka, M. Hashimoto, M. Nishiyama, T. Goto, M. Okuhara, M. Kohsaka, H. Aoki and H. Iminaka. 1987. J. Antibiot. 40: 1249-1255). Furthermore, FK506 is reported to be produced by microorganisms such as Streptomyces tsukubaensis 9993, Streptomyces sp. ATCC 55098, Streptomyces sp. ATCC 53770, Streptomyces kanaymyceticus KCC S-043, and the like (Muramatsu, H., S. I. Mokhtar, M. Katsuoka, and M. Ezaki. 2005. Actinomyetelolgoica. 19:33-39, U.S. Pat. No. 4,894,366). FK520, a structurally similar analog of FK506, exhibits immunosuppressive activity and antifungal activity and it is reported to be produced from Streptomyces hygroscopicus subsp. ascomyceticus ATCC 14891, Streptomyces hygroscopicus subsp. yakusimaensis 7238, Streptomyces tsukubaensis 993, and the like.

In recent years, the biosynthetic genes of the two materials have been cloned from Streptomyces sp. ATCC 53770 and Streptomyces hygroscopicus subsp. ascomyceticus ATCC 14891 (Motamedi, H., and A. Shafiee. 1998. Eur. J. Biochem 256:528-534, Wu, K. L. Chung, W. P. Revill, L. Katz, C. D. Reeves, 2000. Gene 251:81-90), and are similar in arrangement and composition. Currently, FK506 is commercially available as Prograf™, and has been developed as a neurotropic medicine to prevent organ transplant rejection, allergies and nerve cell damage/dysfunction based on the activity of rapamycin, FK520, and their derivatives, which are tricyclo compounds similar to FK506 and bound to FK506 binding proteins (FKBPs) (Hamilton, G. S, and J. P. Steiner 1998. J. Med. Chem. 41:51119-5143. Gold, B. G., 1999. Drug Metab. Rev. 31:649-663. Scheriver, S. L. and G. R. Carbtree. 1995. Harvey Lect. 91:99-114) (US Patent Publication No. 2005-239813, US Patent Publication No. 2005-070468, US Patent Publication No. 2001-050419, and US Patent Publication No. 2002-086015). Research has been conducted to determine a method for improving a culture medium or a culture method affecting the production of FR506 using Streptomyces clavurigerus KCTC 10561BP (Korean Patent No. 485877) and a nutritional factor in Streptomyces sp. MA 6858 affecting the production of FK506 (Yoon, Y. J. and C. Y. Choi. 1997. J. Fermen. Bioengin. 83:599-603). Because purification of tricyclo compounds is generally carried out by collecting mycelial cakes using diatomite and a drum filter from a cultured broth, collecting a target material therefrom using organic solvents, such as methanol or the like, and applying the mixture of the collected solution and the filtered culture broth to a column filled with a synthetic resin (U.S. Pat. No. 4,894,366, Korean Patent No. 485,877, and Japanese Patent Publication No. 1999-0014), the processes become significantly complicated.

### DISCLOSURE

### Technical Problem

FK506 is useful as a medicine for treatment of autoimmune diseases, organ transplant rejection, erythroblastosis, etc. In developing such a medicine, high purity must be ensured. Therefore, it is necessary to establish a microorganism with improved productivity, a fermentation process, and an efficient purification process. An aspect of the present invention is to provide a fermentation method for tricyclo compounds including FK506 and FK520 that ensures an increased yield of tricyclo compounds and permits a simplified process of purifying the same.

### Technical Solution

In accordance with an aspect of the present invention, the method of fermenting tricyclo compounds includes adding a hydrophobic synthetic adsorbent resin capable of adsorbing hydrophobic tricyclo compounds as a carrier to a fermentation medium of actinomyces to prevent the inhibition of production, thereby improving yield of the tricyclo compounds, specifically FK506 and FK520 (Chemistry FIG. 2).

In accordance with another aspect of the present invention, a method of purifying tricyclo compounds includes extracting the tricyclo compounds from a resin added to a fermentation medium.

### Advantageous Effects

According to exemplary embodiments of the present invention, a hydrophobic synthetic adsorbent resin is added for production and purification of hydrophobic tricyclo compounds, thereby substantially improving yield of tricyclo compounds while simplifying purification thereof.

### DESCRIPTION OF DRAWINGS

The above and other objects, features and other advantages of the present invention will be more clearly understood from the following detailed description taken in conjunction with the accompanying drawings, in which:

FIG. 1 is a graph depicting HPLC analysis results of tricyclo compounds, FK506 and FK520.

In an experimental control group to which a resin was not added, analysis was carried out on a whole control culture media, a control supernatant where cells were removed, and control cells that were collected after being centrifuged. In an HP-20 added culture group, analysis was carried out on a whole fermented culture media where an HP-20 synthetic resin was added at 5% (v/v).

FIG. 2 is a graph depicting LC-Mass analysis results of FK506 and FK520.

FK506 [m/z, 822], FK520 [m/z, 810]

FIG. 3 is a graph depicting yields of FK506 and FK520 when using different kinds of synthetic resins.

Various kinds of synthetic resins were added, each at 5% (v/v), to a culture medium. Then, the medium with the resin was sterilized and fermented for six days to measure a yield of FK506 and a yield of FK520. The control group was an experimental group where a synthetic resin was not added.

FIG. 4 is a graph depicting yields of FK506 and FK520 on a daily basis according to days when the resin was added.

The resin (HP-20) was used for experiments. The resin was separately sterilized during culturing and added on different days corresponding to a final concentration of 5% (v/v). The medium was fermented for six days to measure a yield of compounds. The control group is an experimental group to which resin was not added.

FIG. 5 is a graph depicting yields of FK506 and FK520 on a daily basis according to culturing days and resin added days.

A yield of FK506, depending on a culturing day with no resin added, obtained by collecting a small amount of the compound on a predetermined day in fermentation.

A yield of FK506, depending on a culturing day with the resin added, obtained from FIG. 4.

FIG. 6 is graphs depicting yields of FK506 and FK520 according to an added amount of resin.

The added amount of resin is given as a volume ratio. The control group is an experimental group to which resin was not added.

FIG. 7 illustrates results of a sensibility test of actinomyces to FK506. In the test, Streptomyces venezuelae, Streptomyces sp. GT1005 and Streptomyces sp. ATCC 55098 were used as the actinomyces, and FK506, with a concentration displayed on the drawing, was employed.

FIG. 8 is a graph depicting an extraction amount of FK506 and an extraction amount of FK520 according to solvents.

A resin was extracted from the medium to which the resin (HP-20) was added, and a solvent was added thereto at a volume ratio twice that of the resin to extract the compounds. The compounds were quantitatively analyzed.

FIG. 9 is a graph comparing yields of FK506 obtained from recycled resins.

Resin (HP-20) was added at 5% (v/v). A control group was an experiment to which resin was not added. New resin, once-used resin (Re-1), and twice-used resin (Re-2) were used after being recycled by a method mentioned in Example 7.

### BEST MODE

The present invention provides a fermentation method wherein fermentation is performed after adding a hydrophobic synthetic adsorbent resin capable of adsorbing hydrophobic tricyclo compounds as a carrier to a fermentation medium of actinomyces to prevent a reverse reaction, thereby providing an increased yield of tricyclo compounds, specifically FK506 and FK520 (Chemical Formula 2).

Furthermore, the present invention provides a method of purifying tricyclo compounds by extracting the tricyclo compounds from the resin added to a fermentation medium.

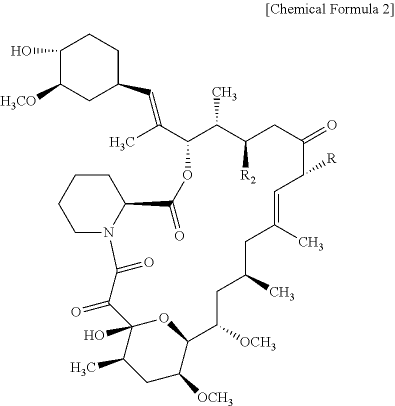

(The above compound is FK506 in case of R═C₃H₅, and FK520 in case of R═C₃H₅)

Next, an exemplary embodiment of the present invention will be described in detail.

The microorganism used in the exemplary embodiment of the present invention is Streptomyces sp. GT1005 which produces both FK506 and FK520. However, the present invention is not limited thereto and can be applied to any microorganism that produces tricyclo compounds, specifically FK506 and/or FK520.

Since tricyclo compounds are hydrophobic, a hydrophobic adsorbent synthetic resin which can be used as a carrier of the tricyclo compounds is added to a fermentation medium. This prevents adsorption of the tricyclo compounds to hydrophobic mycelia to overcome low yields by inhibition of production and improve a yield of the tricyclo compounds.

As a carrier, the hydrophobic absorbent resin may include a synthetic resin comprising styrene/divinylbenzene copolymers or aliphatic ester as a main ingredient, and may be one selected from the group consisting of Diaion HP-20, Amberlite XAD-2 Amberlite XAD-4, Amberlite XAD-7, Amberlite XAD-7HP, Amberlite XAD-8, Amberlite XAD-16, Amberlite XAD-16 HP, Amberlite XAD-1180, Amberlite XAD-2000 and Amberlite XAD-2010.

The added amount of synthetic resin is 1˜15% (v/v), preferably 3˜5% (v/v), but most preferably 5% (v/v). If the synthetic resin is directly added to a culture medium for fermentation, three times or greater increase in yield can be expected.

The synthetic resin can be added to the culture medium at any time from the beginning of culturing to the end of culturing, preferably within three days (0˜72 hours) from the beginning of the culture, which is closely related to production characteristics of the tricyclo compounds. That is, in an experiment in which the resin was not added, a yield of FK506 starts to increase from the second day and reaches a peak on the fifth or sixth day. Thus, the synthetic resin is preferably added at the beginning of culturing to improve the yield of tricyclo compounds.

The tricyclo compounds, specifically FK506 and FK520, are collected from a fermentation medium that has been cultured for four to seven days after the addition of the synthetic resin. Most of the tricyclo compounds produced are adsorbed to the hydrophobic adsorbent synthetic resin added as the carrier (90˜98%). Thus, it is more preferable to collect the compounds from the synthetic resin after separating the synthetic resin from the mycelial cake or the culture medium.

The synthetic resins are collected by centrifugal separation or filtration. Preferably, a fiber or wire sieve with a smaller pore size (average 250 μm) than the size of the synthetic resin (average 250 μm) is used, but it is most preferable to use a wire sieve (with a pore size of 60-250 μm).

A solvent for eluting the tricyclo compounds from the synthetic resin is one selected from the group consisting of methanol, ethanol, acetone, acetonitrile, butanol, ethyl acetate, chloroform, dichloromethane and hexane. Preferably, the solvent is one selected from methanol, ethanol, acetone and acetonitrile, which can be mixed with water and most preferably is one selected from acetone and an acetone aqueous solution (40˜100%).

### MODE FOR INVENTION

Next, examples of the present invention will be described in detail. However, it is apparent to those skilled in the art that the present invention is not limited to these examples of the present invention disclosed below but can be implemented in various ways.

### Example 1

### Fermentation for Production of Tricyclo Compounds

To produce tricyclo compounds, a pre-culture medium (1% soluble starch, 1% glycerol, 2% soy bean flour, 0.2% CaCo₃, and 0.05% GE-304) was inoculated with a mycelial solution of streptomyces sp. GT 1005 and cultured at 27-30° C. for 24 hours. A main culture medium (7% soluble starch, 0.5% soy bean flour, 1.7% yeast extract, 0.1% (NH₄)₂SO₄, 0.5% corn steep liquor, 0.1% CaCO₃, and 0.05% GE-304) was inoculated with 1˜5% of the resultant product and cultured at 27˜30° C. for six days. In Erlenmeyer-flask culturing, both pre-culturing and main culturing were carried out in a 500 ml flask with 30 ml of the medium by a shaking culture at 230 rpm. In 5 l jar fermentor culturing, 3 l of the main-culture medium was used and the culturing was performed at a ventilation rate of 1.5 vvm and 600˜900 rpm, and a hydrophobic absorbent solid resin selected from diaion HP-20, Amberlite XAD4, Amberlite XAD7H, and Amberlite XAD14 was added as a carrier at different times and in different amounts for each example.

### Example 2

### Identification and Quantitative Analysis of Tricyclo Compounds

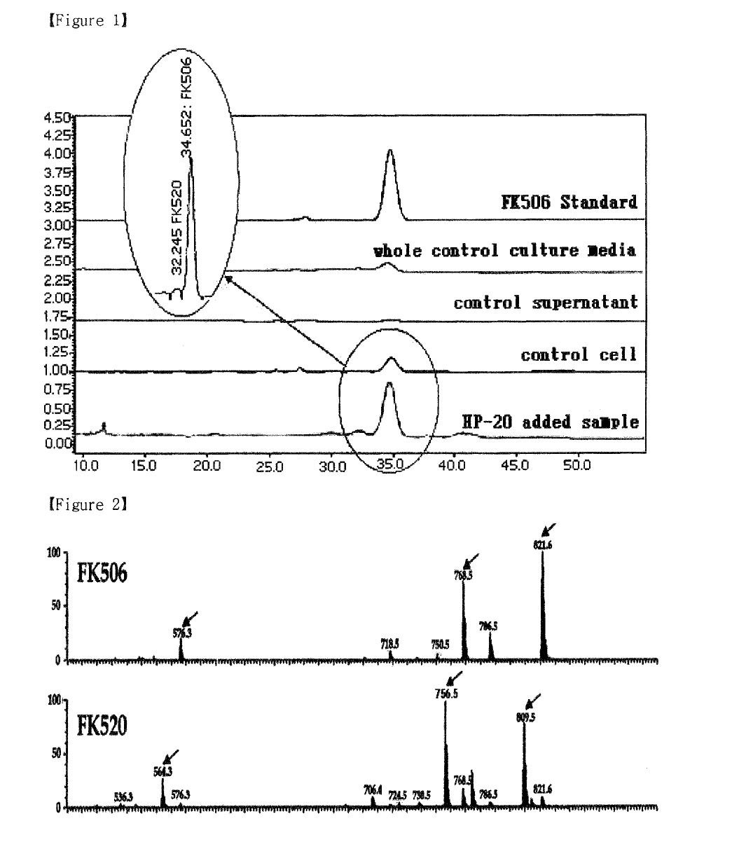

FK506 and FK520 were identified and quantitatively analyzed. FK506 and FK520 standards were purchased from A.G. Scientific, Inc. Molecular weights of FK506 and FK520 standards and the fermented product were identified by LC-ESI-MS/MS (FK506 M/Z, 822; FK520 M/Z, 810). As needed, FK506 and FK520 were identified by a process wherein samples and standards were injected at the same time to examine the same retention times (FK506, 34 minutes; FK520, 33 minutes) (see FIGS. 1 and 2). For the quantitative analysis, high performance liquid chromatography (HPLC) was conducted under the conditions as in Table 1, and samples dissolved in a 50% acetone aqueous solution and having an amount within an effective range (0.1˜1 mg) of the quantitative standard calibration curve were used.

TABLE 1

| Items | Conditions | |
|---|---|---|
| Column | Hypersil GOLD C18 analytical | |
| | column (Thermo) | |
| Temperature of Column | 55 | □ |
| Adsorption wavelength | 210 | nm |
| Mobile Phase | 50% | acetonitrile |
| Flow | 1 | ml/min |
| Inflow of Sample | 20 | μl |

### Example 3

### Increase in Yield According to Solid Resins Used as Carrier

Most of the tricyclo compounds (FK506 and FK520) which were produced were present in mycelia (see FIG. 1). It is assumed that such characteristics of the tricyclo compounds are related with hydrophobic properties of the tricyclo compounds. The biosynthesized hydrophobic tricyclo compounds were adsorbed to a hydrophobic mycelial cake. It was considered that the excessive accumulation of the tricyclo compounds ultimately stops or reduces production of the tricyclo compounds via growth inhibition or feedback inhibition. Therefore, if a hydrophobic solid material having similar properties to those of the mycelial cake was to be added to a fermentation medium, it would serve as a carrier for the tricyclo compounds instead of the mycelial cake. That is, the inventors considered that the produced tricyclo compounds were adsorbed to the hydrophobic solid synthetic resin instead of the mycelial cake when the hydrophobic solid synthetic resin was added to the fermentation medium during fermentation.

As absorbent resin to be added to the fermentation medium, HP-20 (Yiryoong Chemicals Co., Ltd.), Amberlite XAD-4 (Rohm & Hass), Amberlite XAD-7H (Rohm & Hass), and Amberlite XAD-16 (Rohm & Hass) were added at 5% (v/v) to a main-culture medium, and cultured in a 500-ml Erlenmeyer flask containing 30 ml of a medium for six days as in Example 1. Then, the contents of tricyclo compounds, specifically FK506 and FK520, were analyzed as in Example 2.

Table 2 shows yields of tricyclo compounds depending on resins provided as a carrier.

TABLE 2

| | Yield of FK506 | Yield of FK520 |
|---|---|---|
| Experimental Group | (mg/l) | (mg/l) |
| No resin added control | 101.5 (100) | 1.7 (100) |
| HP-20 | 292 (287) | 6.6 (388) |
| XAD-4 | 262.75 (258) | 6.5 (382) |
| XAD-7H | 299.25 (294) | 5.7 (335) |
| XAD-16 | 281.75 (278) | 5.2 (305) |

As can be seen in Table 2 and FIG. 3, in the experimental group where the resin was added to the medium, the yields of the tricyclo compounds, FK506 and FK520, were at least 2.5 times those of the tricyclo compounds in the control group to which the resin was not added. It is considered that FK506 and FK520 are similar in physical and structural properties and in presumed biosynthetic process so that they increase in yield at a similar rate.

### Example 4

### Increase of Yield According to Addition of Solid Synthetic Resin by Time in Fermentation

To determine proper time in adding a solid synthetic resin, an HP-20 resin, as adopted in Example 3, was added at 5% (v/v) to the culture medium at intervals of one day from the beginning of fermentation to one day before the end of fermentation, followed by fermentation and culturing for six days as in Example 1. The contents of tricyclo compounds (FK506 and FK520) were analyzed as in Example 2.

Table 3 illustrates a yield of the compounds according to the addition time of the resin.

TABLE 3

| Day on which resin | Yield of FK506 | Yield of FK520 |
|---|---|---|
| (HP-20) was added | (mg/l) | (mg/l) |
| No resin added control | 102 | 2.3 |
| Day 0 | 356 | 7 |
| Day 1 | 356.5 | 8.7 |
| Day 2 | 337 | 8 |
| Day 3 | 186.5 | 5.5 |
| Day 4 | 117 | 3.3 |
| Day 5 | 81 | 1.6 |

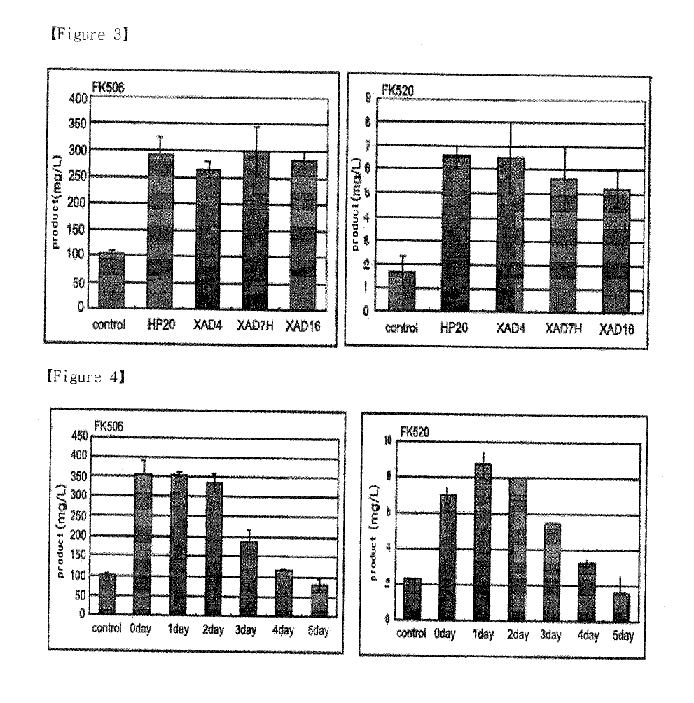

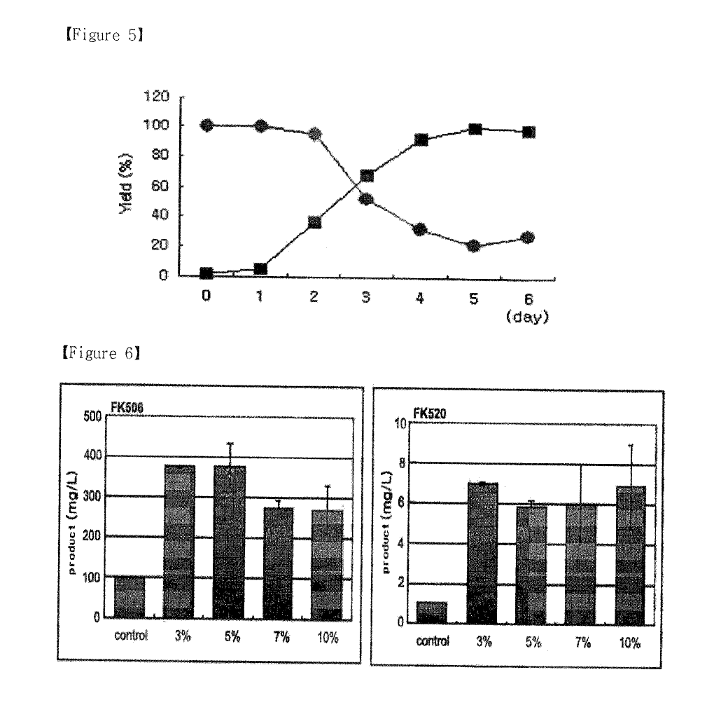

As can be seen from Table 3 and FIG. 4, the compounds in the experimental group where the resin was added at the beginning of cultivation showed the greatest increase in yield. That is, the yield of the compounds in the experimental groups to which the resin was added within two days (48 hours) from the day of the fermentation increased by about three times. The yield began to exponentially decrease after three days. In the experimental group to which the resin was added one day before the end of the fermentation, i.e., five days from the beginning of fermentation, the yield of the compounds hardly increased. This was inversely proportional to the yield curve of FK506 in the control group without the resin added (see FIG. 5). For an increasing yield, in other words, it was effective to add the resin within three days at an initial stage where the production started, but it was useless to add the resin at the stage where the production was completed. Therefore, it was very critical to add a synthetic resin at an initial stage of fermentation. In the following experimental groups of Examples 5 and 6 to which the resin was added, a fermentation medium to which the resin was added was prepared and sterilized, followed by inoculation with mycelia to ensure fermentation starts simultaneous with the addition of a synthetic resin.

### Example 5

### Increase of Yield According to Amount of Solid Resin Used as Carrier

One of synthetic resins, HP20 used in Example 3, was adopted and added to a fermentation medium at 3%, 5%, 7%, and 10% (v/v) as in Example 4, followed by culturing for six days as in Example 1. Tricyclo compounds were extracted by the method of Example 2 and quantities thereof were determined using HPLC.

Table 4 shows a yield according to an added amount of resin.

TABLE 4

| Amount of Resin | Yield of FK506 | Yield of FK520 |
|---|---|---|
| (HP-20) (%) | (mg/l) | (mg/l) |
| 0 | 98 | 1.05 |
| 3 | 377 | 7 |
| 5 | 365.27 | 6.1 |
| 7 | 373.5 | 4 |
| 10 | 207.5 | 4.8 |

As can be seen from Table 4 and FIG. 6, FK506 and FK520 had the greatest yield in the experimental groups where HP20 was added at 3% and 5%, and tended to slightly decrease in yields as the synthetic resin was added at a higher ratio. It is considered that excessive addition of resin has a negative effect upon growth of mycelia.

### Example 6

### Cause of Increase in Yield of Tricyclo Compound by Synthetic Resin

To explain the increase in yield of the compounds by the synthetic resin in detail, the synthetic resin was enclosed with Miracloth (Calbiochem), through which mycelia cannot pass but the medium easily passes, and was added to a fermentation medium, followed by fermentation as in Example 1. As a result, an increase in yields due to the resin did not occur in the experimental group where the synthetic resin was separated and added (Table 5).

TABLE 5

| | Yield of FK506 |
|---|---|
| Experimental Group | (mg/l) (%) |
| No resin added control | 113.0 (100) |
| Resin (HP-20) added group | 313.8 (278) |
| Enclosed Resin (HP-20) added group | 112.6 (100) |

The results show that contact of mycelia with the synthetic resin was important in adsorption of the tricyclo compounds to the synthetic resin. Furthermore, the mechanism of increasing the yield of the tricyclo compounds by the synthetic resin was determined by the fact that tricyclo compounds which would normally absorbed by the mycelium were transferred instead of absorbed by the hydrophobic synthetic resin, so that toxicity or feedback inhibition of the tricyclo compound to the mycelia could be solved, thereby leading to an increase in yield of the tricyclo compounds.

To specifically determine the cause of the increase in yield of the tricyclo compounds, distribution of FK506 and FK520 in the culture broth was examined. HP-20 resin, mycelia, and culture supernatant used for cultivation were separated to analyze the content of tricyclo compounds. As a result, most of the tricyclo compounds (95˜99%) were adsorbed to the HP-20 resin, whereas a very small amount of tricyclo compounds (1˜5%) were present in the mycelia and the supernatant (Table 6).

TABLE 6

| | Yield of FK506 | Yield of FK520 | % |
|---|---|---|---|
| Category | (mg/l) | (mg/l) | (FK506 + FK520) |
| Supernatant | 0 | 0 | 0 |
| Mycelia | 7.2 | 0 | 1.4 |
| HP-20 | 494.5 | 11.8 | 98.6 |

Furthermore, actinomyces were tolerant to an excess (1 mg/ml) of FK506 and growth thereof was not inhibited (see FIG. 7). This result coincides with the report that the tricyclo compounds have antifungal activity and do not have antibacterial activity. It proved that the increase in yield of FK506 and FK520 by the synthetic resin was not obtained by the elimination of their own toxicity. Considering the facts that growth of bacteria was not affected by an excessive amount of tricyclo compounds and that growth rates or weights of cells were not increased by the addition of the resin, it is assumed that yields were not increased by improvement in growth of bacteria via reduction in toxicity of the tricyclo compounds or other secondary metabolites. Therefore, it is one of the reasons for the increased tricycle compounds yield up to remove feedback inhibition material, which is involved in the tricycle compounds yield by tricycle compounds and other hydrophobic secondary metabolites and by the added synthetic resin which entrapped of them. Conclusively, in the present invention where the synthetic resin was added for fermentative cultivation, the tricyclo compounds, specifically FK506 and FK520, adsorbed to mycelia were transferred to an added synthetic resin to prevent feedback inhibition, thereby achieving an increased yield. The present invention enables high tricyclo compound production from a microorganism by overcoming the problems of the conventional technique wherein limited adsorption quantity of tricyclo compounds to mycelia causes the obstacle of development of an microorganism for high tricyclo compound production.

### Example 7

### Collection of Solid Resin, Extraction of FK506 from Solid Resin, and Recycling of Resin

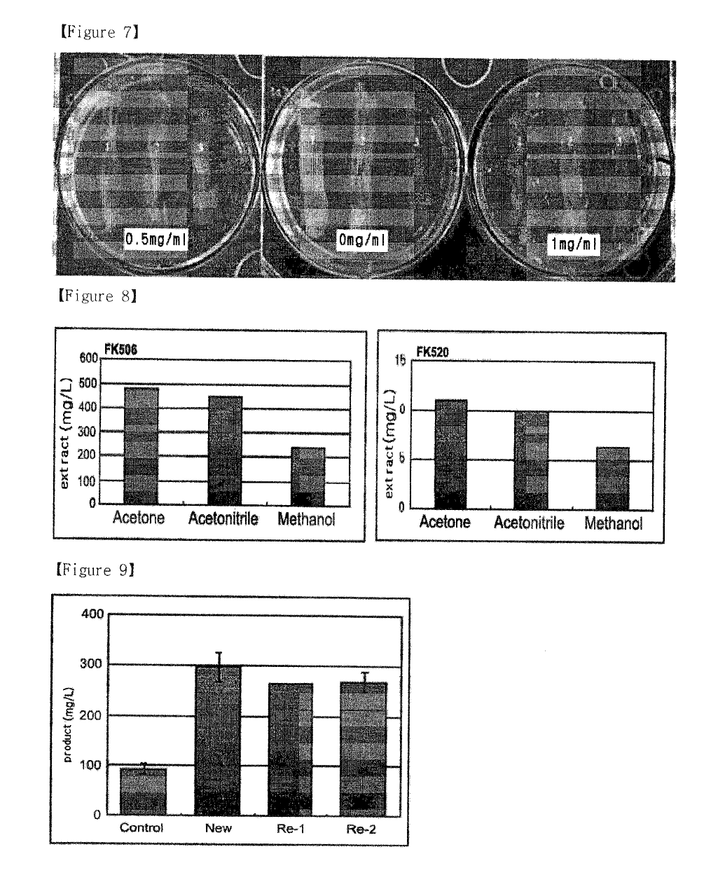

The synthetic resin, to which most of the tricyclo compounds identified in Example 6 were adsorbed, was separated to extract FK506 and FK520 therefrom and to purify the same. Because the synthetic resin (HP-20) has an average size of 250 μm or more, a copper sieve having a pore diameter of 250 μm or less was used to collect the synthetic resin from the culture broth (5 l) after completion of fermentation. The collected synthetic resin was washed with distilled water and filled in a proper-size column. Tricyclo compounds were then extracted from the synthetic resin using various organic solvents, but acetone, methanol, ethanol, and acetonitrile, which can be easily mixed with water, were preferable (Table 7). For acetone, a 50% or more acetone aqueous solution could collect the tricyclo compounds and a 70-75% acetone aqueous solution could collect tricyclo compounds with less impurities and a good yield (see FIG. 8). In extraction with 75% acetone, the resin was washed with a 40% acetone aqueous solution acetone at a volume of three times that of the resin, and the compounds were extracted with a 75% acetone aqueous solution at a volume of three times that of the resin. Here, the collection rate was 94% or more (Table 8). Thus, the fermentation broth with the HP-20 resin added was very effective since the synthetic resin was separated from the culture broth for extraction with an extraction solvent to reduce the amount of extraction solvent while enabling the most of tricyclo compounds to be extracted.

Table 7 shows a collection amount of the compounds from the resin by extraction with a solvent.

TABLE 7

| | Yield of FK506 | Yield of FK520 |
|---|---|---|
| Kind of Resin | (mg/l) | (mg/l) |
| Acetone | 477.5 | 11 |
| Acetonitrile | 448 | 10 |
| Methanol | 237.5 | 6.3 |

Table 8 shows a collection amount of the compounds from the resin by extraction with an acetone aqueous solution.

TABLE 8

| | | | Yield of FK506 | |
|---|---|---|---|---|
| Step | | Volume | (mg) | Yield (%) |
| Sample | Resin (HP-20) | 200 ml | 1491.0 | 100.0 |
| Extraction | 40% acetone | 600 ml | 31.8 | 2.1 |
| Solvent | 75% acetone | 200 ml | 10.3 | 0.6 |
| | | 200 ml | 1193 | 80.0 |
| | | 200 ml | 201.4 | 13.5 |
| | | 200 ml | 0.8 | 0.1 |
| | | 200 ml | 0.0 | 0.0 |

Table 9 shows adsorption efficiency of recycled resin.

TABLE 9

| | Yield of FK506 | Ratio to Control | Ratio to New |
|---|---|---|---|
| | (mg/l) | Group (%) | Resin (%) |
| No Resin Added | 94.5 ± 10.0 | 100.0 | 31.8 |
| Control Group | | | |
| New Resin | 297.0 ± 28 | 314.3 | 100.0 |
| Resin Recycled Once | 264.7 ± 1.25 | 280.2 | 89.1 |
| Resin Recycled Twice | 269.0 ± 19.8 | 284.7 | 90.6 |

It is shown that the adsorption efficiencies of the recycled resins were 90% of the new resin, the collected synthetic resin (HP-20) having been washed with the mixture of 50% isopropyl alcohol and a 50% 1N NaOH aqueous solution, a 4% NaOCl aqueous solution, and distilled water at a quadruple volume ratio each in order (see FIG. 9 and Table 9).

Although the present invention has been described with reference to the embodiments and the accompanying drawings, the present invention is not limited to these embodiments and the drawings. It should be understood that various modifications, additions and substitutions can be made by a person having ordinary knowledge in the art without departing from the scope and spirit of the invention as disclosed in the accompanying claims.

### INDUSTRIAL APPLICABILITY

According to the present invention, hydrophobic tricyclo compounds such as FK506 and FK520 can be yielded up by hydrophobic adsorbent resin which acts as a carrier of them during fermentation, and the resin can be easily harvesting and the compounds can be efficiently extracted from the resin. Therefore, the present invention is useful for efficient production of hydrophobic tricyclo compounds.

1. A method of producing tricyclo compounds using a hydrophobic absorbent resin added to improve yield and purification efficiency of the tricyclo compounds, upon fermentation and purification of a microorganism for producing the tricyclo compounds, the method comprising:
adding a hydrophobic absorbent resin to the microorganism for producing the tricyclo compounds;
collecting the hydrophobic absorbent resin; and
collecting tricyclo compounds from the hydrophobic absorbent resin.

2. The method of producing tricyclo compounds according to claim 1, wherein the tricyclo compounds are at least one of FK506 and FK520.

3. The method of producing tricyclo compounds according to claim 1, wherein the hydrophobic absorbent resin comprises a styrene/divinylbenzene copolymer and an aliphatic ester copolymer as a main ingredient.

4. The method of producing tricyclo compounds according to claim 3, wherein the hydrophobic absorbent resin is at least one selected from the group consisting of Diaion HP-20, Amberlite XAD-2 Amberlite XAD-4, Amberlite XAD-7, Amberlite XAD-7HP, Amberlite XAD-8, Amberlite XAD-16, Amberlite XAD-16 HP, Amberlite XAD-1180, Amberlite XAD-2000, and Amberlite XAD-2010.

5. The method of producing tricyclo compounds according to any one of claim 4, wherein the hydrophobic absorbent resin is 3˜7% (v/v).

6. The method of producing tricyclo compounds according to any one of claim 3, wherein the hydrophobic absorbent resin is 3˜7% (v/v).

7. The method of producing tricyclo compounds according to any one of claim 1, wherein the hydrophobic absorbent resin is 3˜7% (v/v).

8. The method of producing tricyclo compounds according to claim 1, wherein the hydrophobic absorbent resin is at least one selected from the group consisting of Diaion HP-20, Amberlite XAD-2 Amberlite XAD-4, Amberlite XAD-7, Amberlite XAD-7HP, Amberlite XAD-8, Amberlite XAD-16, Amberlite XAD-16 HP, Amberlite XAD-1180, Amberlite XAD-2000, and Amberlite XAD-2010.

9. The method of producing tricyclo compounds according to claim 1, wherein the hydrophobic absorbent resin is added within three days after culturing starts.

10. The method of producing tricyclo compounds according to claim 1, wherein, in the collecting of the tricyclo compounds from the hydrophobic absorbent resin, an effluent used for collecting the tricyclo compounds comprises at least one selected from the group consisting of acetone, methanol, ethanol, acetonitrile, ethyl acetate, hexane, chloroform, and dichloromethane.

11. The method of producing tricyclo compounds according to claim 10, wherein the effluent comprises a 50˜100% (v/v) acetone aqueous solution.

12. The method of producing tricyclo compounds according to claim 1, wherein the hydrophobic absorbent resin is recycled.

13. The method of producing tricyclo compounds according to claim 12, wherein the hydrophobic absorbent resin is washed with a mixture solvent of 50% isopropyl alcohol and a 50% 1N NaOH aqueous solution, a 4% NaOCL aqueous solution, and distilled water in order.
